(19) United States
(12) Patent Application Publication
(10) Pub. No.: US 2007/0185359 A1
(43) Pub. Date: Aug. 9, 2007

(54) Gasoline production by olefin polymerization

(75) Inventors: Benjamin S. Umansky, Fairfax, VA (US); Arthur P. Werner, Alexandria, VA (US); Amanda K. Miller, Fairfax, VA (US); Tomas R. Melli, Haymarket, VA (US); Christopher M. Dean, Chantilly, VA (US); Niveen S. Ismail, Yardley, PA (US); Garland B. Brignac, Clinton, LA (US)

Correspondence Address: ExxonMobil Research & Engineering Company, P.O. Box 900, 1545 Route 22 East, Annandale, NJ, 08801-0900, US

(73) Assignee: ExxonMobil Research and Engineering Company, Annandale, NJ (US)

(21) Appl. No.: 11/698,813

(22) Filed: Jan. 29, 2007

Related U.S. Application Data
(60) Provisional application No. 60/765,184, filed on Feb. 6, 2006.

(51) Int. Cl. C07C 2/02; B01J 8/04

(52) U.S. Cl. 585/517; 422/190

(57) ABSTRACT

A process unit for the zeolite-catalyzed conversion of light refinery olefins from an FCC unit such as ethylene, propylene, and butylene to gasoline boiling range motor fuels comprises at least two sequential, serially connected reactors connected in parallel to a fractionation section with at one or two fractionators for separating the reactor effluents into product fraction with an optional recycle stream or streams. The configurations according to this scheme allow the adjustment of reactor temperature and/or pressure and/or space velocity to be based on the reactivities of the olefin compounds present in the LPG streams so that the gasoline produced in each reactor will be separated immediately, to reduce over-polymerization of the gasoline in the low severity reactor and to ensure that gasoline formed in the low severity reactor will not be sent to the higher severity reactor e.g. with a higher reactor temperature, where excessive polymerization to undesirable higher molecular with products may take place.

### CROSS REFERENCE TO RELATED APPLICATIONS

This application claims priority of U.S. Application Ser. No. 60/765,184, filed 6 Feb. 2006; it is also related to U.S. application Ser. No. 11/362,257, filed 27 Feb. 2006 claiming priority from Ser. No. 60/656,954, filed 28 Feb. 2005, entitled “Gasoline Production By Olefin Polymerization”.

### FIELD OF THE INVENTION

This invention relates to light olefin polymerization for the production of gasoline boiling range motor fuel.

### BACKGROUND OF THE INVENTION

Following the introduction of catalytic cracking processes in petroleum refining in the early 1930s, large amounts of olefins, particularly light olefins such as ethylene, propylene, butylene, became available in copious quantities from catalytic cracking plants in refineries. While these olefins may be used as petrochemical feedstock, many conventional petroleum refineries producing petroleum fuels and lubricants are not capable of diverting these materials to petrochemical uses. Processes for producing fuels from these cracking off gases are therefore desirable and from the early days, a number of different processes evolved. The early thermal polymerization process was rapidly displaced by the superior catalytic processes of which there was a number. The first catalytic polymerization process used a sulfuric acid catalyst to polymerize isobutene selectively to dimers which could then be hydrogenated to produce a branched chain octane for blending into aviation fuels. Other processes polymerized isobutylene with normal butylene to form a co-dimer which again results in a high octane, branched chain product. An alternative process uses phosphoric acid as the catalyst, on a solid support and this process can be operated to convert all the C₃ and C₄ olefins into high octane rating, branched chain polymers. This process may also operate with a C₄ olefin feed so as to selectively convert only isobutene or both n-butene and isobutene. This process has the advantage over the sulfuric acid process in that propylene may be polymerized as well as the butenes and at the present time, the solid phosphoric acid [SPA] polymerization process remains the most important refinery polymerization process for the production of motor gasoline.

In the SPA polymerization process, feeds are pretreated to remove hydrogen sulfide and mercaptans which would otherwise enter the product and be unacceptable, both from the view point of the effect on octane and upon the ability of the product to conform to environmental regulations. Typically, a feed is washed with caustic to remove hydrogen sulfide and mercaptans, after which it is washed with water to remove organic basis and any caustic carryover. Because oxygen promotes the deposition of tarry materials on the catalyst, both the feed and wash water are maintained at a low oxygen level. Additional pre-treatments may also be used, depending upon the presence of various contaminants in the feeds. With the most common solid phosphoric acid catalyst, namely phosphoric acid on kieselguhr, the water content of the feed needs to be controlled carefully because if the water content is too high, the catalyst softens and the reactor may plug. Conversely, if the feed is too dry, coke tends to deposit on the catalyst, reducing its activity and increasing the pressure drop across the reactor. As noted by Henckstebeck, the distribution of water between the catalyst and the reactants is a function of temperature and pressure which vary from unit to unit, and for this reason different water concentrations are required in the feeds to different units. Petroleum Processing Principles And Applications, R. J. Hencksterbeck McGraw-Hill, 1959.

There are two general types of units used for the SPA process, based on the reactor type, the unit may be classified as having chamber reactors or tubular reactors. The chamber reactor contains a series of catalyst beds with bed volume increasing from the inlet to the outlet of the reactor, with the most common commercial design having five beds. The catalyst load distribution is designed to control the heat of conversion.

Chamber reactors usually operate with high recycle rates. The recycle stream, depleted in olefin content following polymerization, is used to dilute the olefin at the inlet of the reactor and to quench the inlets of the following beds. Chamber reactors usually operate at pressure of approximately 3500-5500 kPag (about 500-800 psig) and temperature between 180° to 200° C. (about 350°-400° F.). The conversion, per pass of the unit, is determined by the olefin specification in the LPG product stream. Fresh feed LHSV is usually low, approximately 0.4 to 0.8 hr⁻¹. The cycle length for chamber reactors is typically between 2 to 4 months.

The tubular reactor is basically a shell-and-tube heat exchanger in which the polymerization reactions take place in a number of parallel tubes immersed in a cooling medium and filled with the SPA catalyst. Reactor temperature is controlled with the cooling medium, invariably water in commercial units, that is fed on the shell side of the reactor. The heat released from the reactions taking place inside the tubes evaporates the water on the shell side. Temperature profile in a tubular reactor is close to isothermal. Reactor temperature is primarily controlled by means of the shell side water pressure (controls temperature of evaporation) and secondly by the reactor feed temperature. Tubular reactors usually operate at pressure between 5500 and 7500 kPag (800-1100 psig) and temperature of around 200° C. (about 400° F.). Conversion per pass is usually high, around 90 to 93% and the overall conversion is around 95 to 97%. The space velocity in tubular reactors is typically high, e.g., 2 to 3.5 hr⁻¹ LHSV. Cycle length in tubular reactors is normally between 2 to 8 weeks.

For the production of motor gasoline only butene and lighter olefins are employed as feeds to polymerization processes as heavier olefins up to about C₁₀ or C₁₁ can be directly incorporated into the gasoline. With the SPA process, propylene and butylene are satisfactory feedstocks and ethylene may also be included, to produce a copolymer product in the gasoline boiling range. Limited amounts of butadiene may be permissible although this diolefin is undesirable because of its tendency to produce higher molecular weight polymers and to accelerate deposition of coke on the catalyst. The process generally operates under relatively mild conditions, typically between 150° and 200° C., usually at the lower end of this range between 150° and 180° C., when all butenes are polymerized. Higher temperatures may be used when propylene is included in the feed. In a well established commercial SPA polymerization process, the olefin feed together with paraffinic diluent, is fed to the reactor after being preheated by exchange with the reaction effluent.

The solid phosphoric acid catalyst used is non-corrosive, which permits extensive use of carbon steel throughout the unit. The highest octane product is obtained by using a butene feed, with a product octane rating of [R+M]/2 of 91 being typical. With a mixed propylene/butene feed, product octane is typically about 91 and with propylene as the primary feed component, product octane drops to typically 87.

In spite of the advantages of the SPA polymerization process, which have resulted in over 200 units being built since 1935 for the production of gasoline fuel, a number of disadvantages are encountered, mainly from the nature of the catalyst. Although the catalyst is non-corrosive, so that much of the equipment may be made of carbon steel, it does lead it to a number of drawbacks in operation. First, the catalyst life is relatively short as a result of pellet disintegration which causes an increase in the reactor pressure drop. Second, the spent catalyst encounters difficulties in handling from the environmental point of view, being acidic in nature. Third, operational and quality constraints limit flexible feedstock utilization. Obviously, a catalyst which did not have these disadvantages would offer considerable operating and economic advantages.

The Mobil Olefins-to-Gasoline [MOG] process employs a proprietary shape selective zeolite catalyst in a fluidized bed reactor to produce high octane motor gasoline by the conversion of reactive olefins such as ethylene and propylene in FCC off-gas; butenes as well as higher olefins may also be included and converted to form a high octane, branched chain gasoline product. The feed is converted over the catalyst into C₅+ components by mechanisms including oligomerization, carbon number redistribution hydrogen transfer, aromatization, alkylation and isomerization. Based on olefins converted, MOG yields 60 to 75 weight percent of high-octane gasoline blend stock with specific qualities of the product depending of the processing severity selected and the character of the feed olefins. Typically, the octane rating for the product is in the range of 88 to 91 [R+M]/2. The zeolite catalyst used in the process is environmentally safe and its attrition rate is low, and as an alternative to disposal, the spent catalyst can be reused in the FCC unit to increase octane quality.

The MOG process has, however, the economic disadvantage relative to the SPA process in that new capital investment may be required for the fluidized bed reactor and regenerator used to operate the process. If an existing SPA unit is available in the refinery, it may be difficult to justify replacement of the equipment in spite of the drawbacks of the SPA process, especially in view of current margins on fuel products. Thus, although the MOG process is technically superior, with the fluidized bed operation resolving heat problems and the catalyst presenting no environmental problems, displacement of existing SPA polymerization units has frequently been economically unattractive. What is required, therefore, is an economically attractive alternative to the SPA process for the condensation of light olefins to form motor fuels. Desirably, the process should be capable of operation in existing refinery equipment, especially as a “drop in” type replacement for the solid phosphoric acid catalyst used in the SPA process so that existing SPA polymerization units can be directly used with the new catalyst. This implies that the process should use a non-corrosive, solid catalyst in fixed bed catalyst operation. Furthermore, the catalyst should present fewer handling, operational and disposal problems than solid phosphoric acid and, for integration into existing refineries, the product volumes and distributions should be comparable to those of the SPA process.

Co-pending U.S. patent application Ser. No. 11/362,257, above, describes an improved process for converting refinery olefins to gasoline products. The process uses a zeolite polymerization catalyst which can be used on a direct, drop-in basis for the SPA catalyst of the conventional polygas units. As described in that application, the process unit for the improved process utilizes the reactor of an existing SPA unit with the SPA catalyst replaced by the zeolite catalyst. The reactor is a single reactor with recycle supplied as quench in order to moderate the exotherm resulting from the polymerization reaction.

Although the configuration for the process unit described in Ser. No. 11/362,257 produces good quality gasoline boiling range product of excellent quality, it is desirable to achieve certain operational advantages which are not readily attainable with the single-reactor unit configuration. One problem which is encountered with single-reactor operation is that the different olefins in the FCC off-gas streams used as feeds have differing reactivities in polymerization reactions and therefore require different reactions conditions for optimal conversion. Among the isomeric butenes, for example, iso-butylene is the most reactive isomer and can be readily polymerized to C₈ products over a zeolite catalyst. The 2-butene isomers (cis- and trans-) by contrast, are the most difficult to polymerize, requiring higher reactor temperatures and pressures while 1-butene occupies an intermediate position. The differing reactions severities required for optimal or even acceptable levels of conversion for all the olefins in the FCC gas streams cannot be attained in a single reactor configuration. The term “polymerized” is used in this specification together with its cognates in a manner consistent with the petroleum refinery usage although, in fact, the process is one of oligomerization (which term will be used in this specification interchangeably with the conventional term) in which a low molecular weight liquid polymer (oligomer) is the desired product.

The present invention provides an improved configuration or set of unit configurations which enable the different olefins in refinery streams to be converted effectively to gasoline range products with reduced levels of undesirable high boiling range materials.

### SUMMARY OF THE INVENTION

According to the present invention, the process unit for the zeolite-catalyzed conversion of light olefins such as ethylene, propylene, and butylene to gasoline boiling range motor fuels comprises at least two sequential, serially connected reactors connected to a fractionation section or one or more, usually two, two fractionators for separating the reactor effluents into product fractions with an optional recycle stream or streams. Variant configurations according to this general scheme are described in detail below. Advantages of the new configurations are as follows:
1. They allow the adjustment of reactor temperature and/or pressure and/or space velocity to be based on the reactivities of the olefin compounds present in the LPG streams. Accordingly, the most reactive compounds such as iso-butene will react in a low severity reactor and the less reactive compounds such as 1-butene will react in a subsequent reactor with higher severity.
2. Gasoline produced in each reactor will be separated immediately. This will reduce over-polymerization of the gasoline in the low severity reactor and gasoline formed in the low severity reactor, for example, will not be sent to the reactor with a higher reactor temperature where additional polymerization to undesirable higher molecular weight products might take place.
3. Improved product quality, yield and catalyst life by adaption of the process conditions to catalyst needs.
4. The first (low severity) reactor(s) can act as guard bed(s) in case that an upset takes place upstream which sends feed contaminants to the unit.
5. Conversion in each reactor can be adjusted according to catalyst life requirement or process conditions. The increase or decrease in reactor severity of operating conditions will adjust the conversion value of the reactors.
6. These configurations can be used for new grass root units, or for retrofitting existent polygas or other available units in the refinery. A retrofit example could include a refinery having an MTBE unit followed by a polygas unit (or alkylation unit) that can easily be converted to the new configuration.
7. In the new configurations, the equipment types and number of reactors and separation towers do not change substantially from the traditional configuration of polygas units. Capital investment for grass root units will be similar to the traditional configuration of polygas units

The preferred catalysts for use in the present process as a direct drop-in replacement for the solid phosphoric acid catalyst in conventional SPA process units is a solid, particulate catalyst which is non-corrosive, which is stable in fixed bed operation, which exhibits the capability of extended cycle durations before regeneration is necessary and which can be readily handled and which can be finally disposed of simply and economically without encountering significant environmental problems. These catalysts comprise a member of the MWW family of zeolites, a family which includes zeolites PSH 3, MCM-22, MCM 49, MCM 56, SSZ 25, ERB-1 and ITQ-1. It is, however, possible to use alternative zeolites which are active for olefin polymerization, as noted below.

The products from the molecular sieve catalysts are notably superior as motor gasolines to the products produced with the SPA catalysts in excellent yields. The gasoline boiling range [C₅+-200° C.] [C₅+-400° F.] products from the molecular sieve process using a propylene feed under appropriate conditions are achieved in very high yields while the C₅-C₁₂ yield is at least 95%, indicating an excellent yield in the most useful portion of the gasoline boiling range with very little of the environmentally problematical heavier components. The ignition qualities of the gasoline product are also excellent as a result of a high degree of chain branching in the product which is free of aromatics and therefore very acceptable from the environmental point of view.

The unit configurations described above take advantage of the reactivity differences of the olefin compounds contained in LPG feed for dimerization or trimerization reactions (condensation reactions). By having two sets of reactors operating at different severities (e.g. different temperature/similar pressure) formation of the gasoline range product from the different olefins in each reactor is favored. Interstage separation of the product gasoline in the fractionation section means that the initial polymerization products (dimer or trimer) will not be exposed to the higher temperatures associated with higher severity operation leading to the formation of heavy polymer, improving gasoline properties and yields, and extending catalyst cycle life. Units with these process configurations can be used to produce jet and distillate boiling range products. To do this, the severity of the reactors can be increased and/or part of the bottoms product of the fractionation tower can be recycled back to the reactors for additional reaction. In processes of this type, an additional fractionation column may be used to separate the gasoline, jet and/or distillate products.

### DRAWINGS

FIG. 1 shows a process schematic for an olefin polymerization unit for converting light refinery olefins to motor gasoline with two serially connected reactors and a fractionation section comprising a common fractionator.

FIG. 2 shows a process schematic for an olefin polymerization unit for converting light refinery olefins to motor gasoline with two serially connected reactors and a fractionation section comprising a common fractionator which supplies recycle to the first reactor.

FIG. 3 shows a process schematic for an olefin polymerization unit for converting light refinery olefins to motor gasoline with two serially connected reactors and a fractionation section comprising two fractionators.

FIG. 4 shows a process schematic for an olefin polymerization unit for converting light refinery olefins to motor gasoline with two serially connected reactors and a fractionation section comprising two fractionators with the second fractionator supplying recycle to the second reactor.

FIG. 5 shows a process schematic for an olefin polymerization unit for converting light refinery olefins to motor gasoline with two serially connected reactors and a fractionation section comprising two fractionators, each supplying recycle to its own associated reactor.

### DETAILED DESCRIPTION OF THE INVENTION

### Catalyst, General Process Conditions

The preferred catalysts used in the present process contain, as their essential catalytic component, a molecular sieve of the MWW type. A complete description of this class of catalysts which is found in application Ser. No. 11/362,257 to which reference is made for a description of the useful catalysts and their general mode of use and the process conditions applicable to their use. It is, however, possible to use alternative zeolites which are active for olefin polymerization, including intermediate pore size zeolites such as ZSM-5, ZSM-11 including the relatively large pore material within this family, ZSM-12, and the constrained intermediate pore size zeolites ZSM-22, ZSM-23 and ZSM-35. The preferred zeolites are the members of the MCM-22 family, including MCM-22 itself and MCM-49.

### Olefin Feed

The olefin feeds which may be used in the present process units are normally obtained by the catalytic cracking of petroleum feedstocks to produce gasoline as the major product. A complete description of suitable feeds is found in application Ser. No. 11/362,257, to which reference is made for a description of them and of the process conditions applicable to their use.

### Process Parameters

The general process parameters are as described in application Ser. No. 11/362,257, to which reference is made for a description of them. In brief, the present process is notable for its capability of being operated at low temperatures and under moderate pressures. In general, the temperature will be from about 120° to 250° C. (about 250° to 480° F.) and in most cases between 150° and 200° C. (about 300°-390° C.). Temperatures of 170° to 180° C. (about 340° to 360° F.) will normally be found optimum for feeds comprising butene while higher temperatures will normally be appropriate for feeds with significant amounts of propene. For the dimerization of isobutene and/or 1 butene and/or propylene, reactor temperature will be between approximately 20° C. to 150° C. with the LHSV between approximately 0.5 to 10 hr⁻¹. Pressures may be those appropriate to the type of unit from which the conversion was made, so that pressures up to about 7500 kPag (about 1100 psig) will be typical but normally lower pressures will be sufficient, for example, below about 7,000 Kpag (about 1,000 psig) and lower pressure operation may be readily utilized, e.g. up to 3500 kPag (about 500 psig). Ethylene, again, will require higher temperature operation to ensure that the products remain in the gasoline boiling range. Space velocity may be quite high, for example, up to 50 WHSV (hr−1) but more usually in the range of 5 to 30 WHSV.

The second reactor in the sequence is operated at higher severity in comparison with the first reactor in order to convert the unreacted olefins which have passed through the first reactor. Normally, higher severity may be provided by the use of higher temperature and/or higher pressure by heating the feed to the second reactor or with recompression but other expedients which are more effective for converting the more refractory olefins may be utilized. For example, as the volume of olefin passing through the second reactor is less than that passing through the first, a decrease in space velocity is inherently attained with its potential for increased yield as a result of longer catalyst contact time. Equally, a catalyst which is more effective for the polymerization of the more refractory olefins may be used to provide effective higher severity operation.

### Process Unit Configurations

The configurations envisaged according to the present invention can be categorized conveniently as follows:

Twin Reactor Sequential, One Fractionator

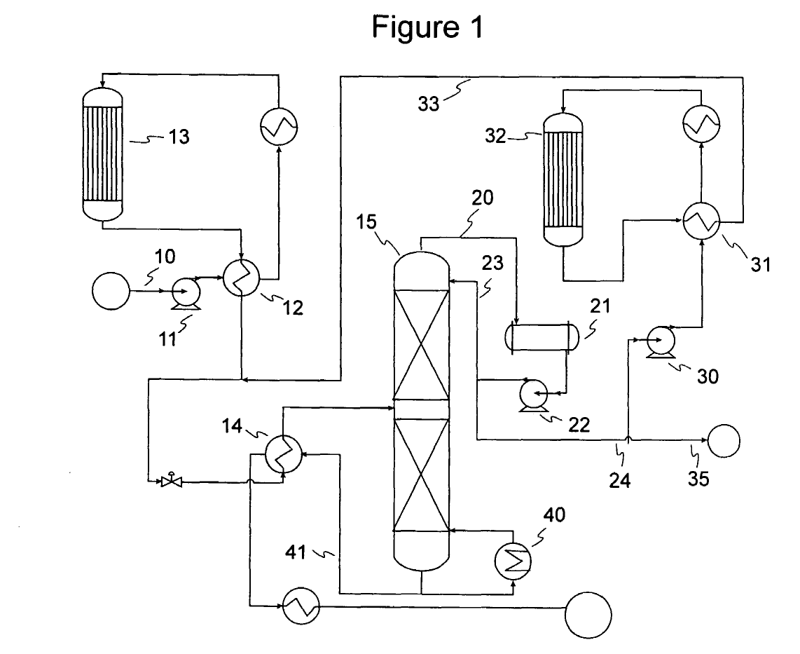

No recycle—FIG. 1

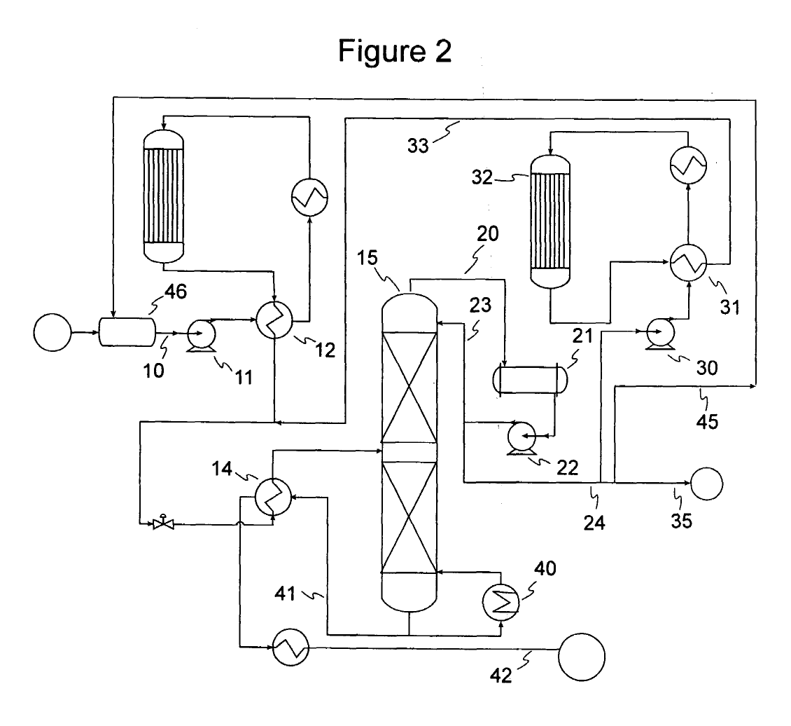

Recycle to first reactor (lower severity)—FIG. 2

Twin Reactor Sequential, Two Fractionators

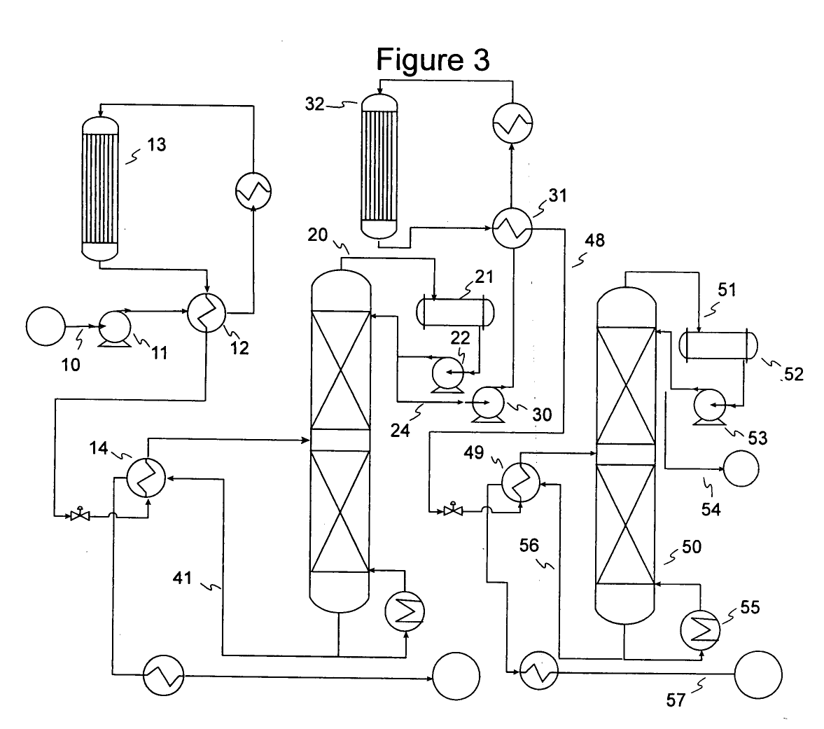

No recycle—FIG. 3

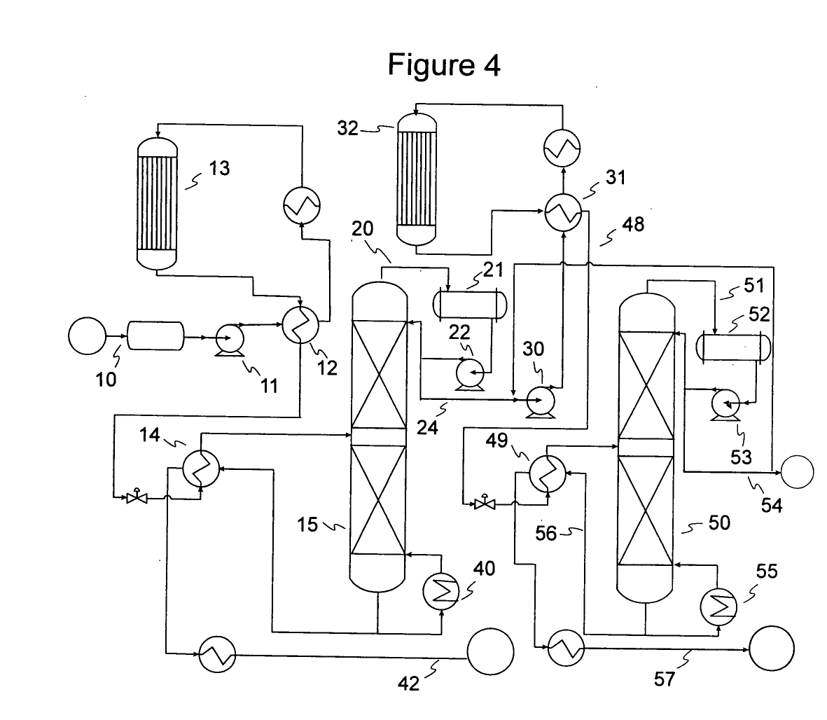

Recycle to second reactor only—FIG. 4

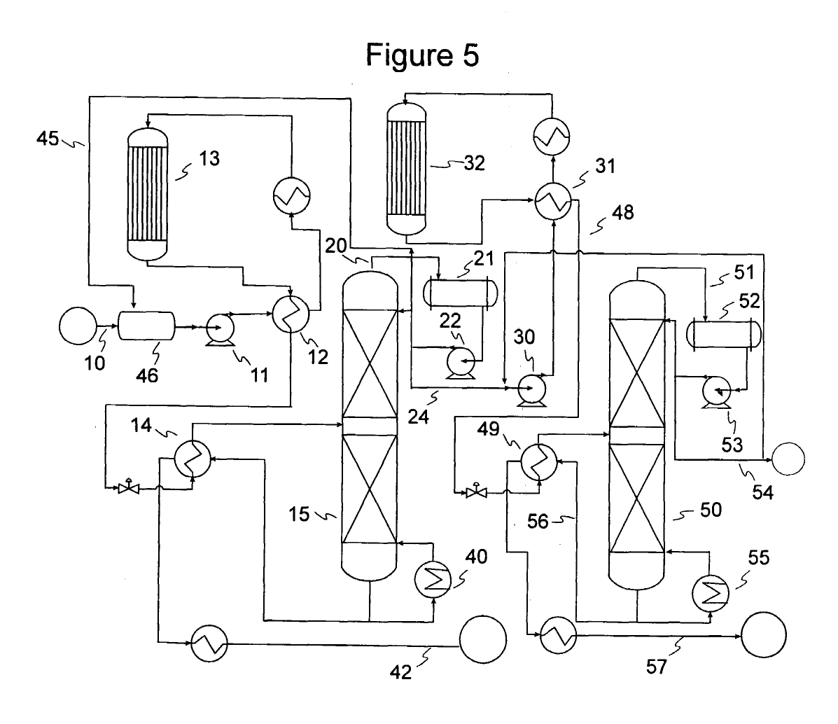

Recycle to both reactors—FIG. 5.

The process unit shown in FIG. 1 utilizes two reactors for the attainment of optimal reaction conditions in each reactor. A shared fractionator is used and no recycle is provided. Olefin LPG feed enters the unit through line 10 before passing successively through compressor 11 and effluent heat exchanger 12 before entering reactor 13 for polymerization to form gasoline product. From the reactor, the effluent passes through heat exchangers 12 and 14 before entering common fractionator 15 which separates the light fraction from the heavy fraction. The light fraction is taken off through overhead 20 to drum 21 and then by way of pump 22 is divided with a portion entering fractionator tower 15 as reflux and another portion being taken as second stage feed through line 24 to pump 30 and second stage effluent heat exchanger 31 to reactor 32 in which a second step of polymerization is carried out, usually under conditions of greater severity so as to polymerize the less reactive olefins e.g. ethylene, which pass through the first stage reactor. The effluent from the second stage reactor passes through line 33 to join the first stage effluent in passing through heat exchanger 14 to the fractionator. Excess unreacted light gas is vented through line 35. Heavy product, including the desired gasoline fraction is withdrawn from the bottom of fractionator 15 by way line 41 with reboil passing in a loop including heat exchanger 40. After passing through effluent heat exchanger 14, the product including the polymerized gasoline leaves the unit through line 42.

Although not shown in the figure, the use of a guard bed ahead of the catalyst bed in the in the first reactor is particularly desirable since the refinery feeds customarily routed to polymerization units (as distinct from petrochemical unit feeds which are invariably high purity feeds for which no guard bed is required) may have a contaminant content, especially of polar catalyst poisons such as the polar organic nitrogen and organic sulfur compounds, which is too high for extended catalyst life. The guard bed may be maintained in a separate vessel ahead of the first reactor in order to allow for replacement or regeneration of the guard bed catalyst. In swing cycle operation, the guard bed may be operated on a swing cycle with two beds, one bed being used on stream for contaminant removal and the other on regeneration in the conventional manner. If desired, a three-bed guard bed system may be used with the two beds used in series for contaminant removal and the third bed on regeneration. With a three guard bed system used to achieve low contaminant levels by the two-stage series sorption, the beds will pass sequentially through a three-step cycle of: regeneration, second bed sorption, first bed sorption.

The catalyst used in the guard bed will often be the same catalyst used in the polymerization reactors as a matter of operating convenience but this is not required: if desired another catalyst or sorbent to remove contaminants from the feed may used, typically a cheaper guard bed sorbent, e.g a used catalyst from another process or alumina. Because the objective of the guard bed is to remove the contaminants from the feed before the feed comes to the reaction catalyst and provided that this is achieved, there is wide variety of choice as to guard bed catalysts and conditions useful to this end. The volume of the guard bed will normally not exceed about 20% of the first catalyst bed volume.

The unit shown in FIG. 2 is similar to that of FIG. 1 (with similar parts numbered accordingly) except that recycle is provided in the form of the light fraction from fractionator 15 with this stream passing through first stage recycle line 45 to feed drum 46 at which point it re-enters the system. The light recycle stream will comprise mainly light paraffins from the LPG feed which have, of course, not undergone reaction over the catalyst. The increased volume of inerts therefore mitigates the exotherm in each reactor although at the cost of reduced unit capacity.

The unit shown in FIG. 3 is similar to that of FIG. 1 (with similar parts numbered accordingly) as far as the second reactor. From the second reactor, 32, however, the effluent passes from heat exchanger 31 through line 48 to second fractionator 50 by way of heat exchanger 49. The light ends are taken out through overhead 51 into the reflux loop with its associated drum 52 and reflux pump 53. Excess unreacted LPG is vented through line 54. The heavy ends from second fractionator 50 are taken from the bottom of the tower with reboil provided by heat exchanger 55. Heavy product gasoline is taken out of the unit by way of heat exchanger 49, exchanging heat with incoming effluent from second reactor 32 in line 48 before leaving through line 57. This unit configuration permits reaction parameters in both reactors to be more closely controlled by appropriate choice of flow rates to the individual reactors.

The configuration shown in FIG. 4 is similar to that of FIG. 3 (with similar parts numbered accordingly) except that in this case, recycle is provided to the second reactor. The recycle stream comprises a light paraffinic stream which is taken from line 54 and returned to the inlet of second stage reactor feed pump 30, to enter with the second stage feed coming from the first fractionator. The use of the recycle stream to the second reactor permits greater control over the second stage polymerization reaction; operation otherwise is in the same manner as with FIG. 3.

The configuration shown in FIG. 5 is a hybrid of those shown in FIGS. 2 and 4 (with similar parts numbered accordingly). In this case, recycle is provided to the first reactor by return of first stage light product from the overhead from fractionator 15 which is conducted through first stage recycle line 45 to the first stage feed drum 46; second stage recycle is provided from the second stage fractionator overhead taken from line 54 to second stage feed pump 30. This configuration provides the maximum of operational flexibility in enabling the optimum reaction conditions to be selected individually for each reactor.

The reactors in these configurations can be tubular, chamber, or a combination of both. In the configurations described above, only two reactors are shown to illustrate the principles by which the reactors and fractionators can be combined. In practice, each reactor could represent several reactors operating in parallel trains at similar operating conditions. In addition, the principles applicable to two-reactor and/or two-fractionator operation can be extended to operation with three or more sequential reactors with fractionators associated with the reactors according to the above schemes, although economics and diminishing returns will normally militate against this degree of complication.

The reactors themselves can be chamber type or tubular type and may conveniently be SPA unit conversions made according to the principles set out in application Ser. No. 11/362,257.

### Gasoline Product Formation

With gasoline as the desired product, a high quality product is obtained from the polymerization step, suitable for direct blending into the refinery gasoline pool after fractionation as described in application Ser. No. 11/362,257. With clean feeds, the product is correspondingly low in contaminants. The product is high in octane rating with RON values of 95 being regularly obtained and values of over 97 being typical; MON is normally over 80 and typically over 82 so that (RON+MON)/2 values of at least 89 or 90 are achievable with mixed propylene/butene feeds. Of particular note is the composition of the octenes in the product with a favorable content of the higher-octane branched chain components. The linear octenes are routinely lower than with the SPA product, typically being below 0.06 wt. pct. except at the highest conversions and even then, the linears are no higher than those resulting from SPA catalyst. The higher octane di-branched octenes are noteworthy in consistently being above 90 wt. pct., again except at the highest conversions but in all cases, higher than those from SPA; usually, the di-branched octenes will be at least 92 wt. pct of all octenes and in favorable cases at least 93 wt. pct. The levels of tri-branched octenes are typically lower than those resulting from the SPA process especially at high conversions, with less than 4 wt. pct being typically except at the highest conversions when 5 or 6 wt. pct. may be achieved, approximately half that resulting from SPA processing. In the C5-200° C. product fraction, high levels of di-branched C8 hydrocarbons may be found, with at least 85 weight percent of the octene components being di-branched C₈ hydrocarbons, e.g. 88 to 96 weight percent di-branched C8 hydrocarbons.

Depending on feed composition, reactions other than direct olefin polymerization may take place. If branch chain paraffins are present, for example, olefin-isoparaffin alkylation reactions may take place, leading to the production of branched-chain, gasoline boiling range products of high octane rating. The reaction between butene and iso-butane and between propylene and iso-butane is of particular value in the product of very desirable, high octane gasoline components. At low to moderate olefin conversion levels, the isoparaffin-olefin alkylation reaction is not significant but at higher conversions above about 75% (olefin conversion), this reaction will increase markedly with the production of high octane gasoline components.

The table below shows the most important parameters of process simulations performed to compare the traditional polygas unit with the proposed configurations. The table shows that overall conversion was maintained between 90 to 95 in all the cases. The feed used in the simulation represents a typical LPG feed containing C₃ and C₄ olefins such as could be obtained from FCC, steam cracking, coking, hydrocracking or other refinery process units. The configuration can be used for C₃ feeds, C₄ feed, or a combination of both.

| | | |
|---|---|---|
| | LPG | |
| Feed | Product | Gasoline |

| | | Rate | % | Rate | % | Product | Olefin |
|---|---|---|---|---|---|---|---|
| Fig | Configuration | (B/D) | Olefins | (B/D) | Olefins | Rate (B/D) | Conv. |
| 1 | 2Rx-1 Frac-No Recycle | 7300 | 70% | 2223 | 14% | 3879 | 94% |
| 2 | 2Rx-1 Frac-1 Recycle | 7300 | 70% | 2374 | 15% | 3867 | 93% |
| 3 | 2Rx-2 Frac-No Recycle | 7300 | 70% | 2613 | 20% | 3780 | 90% |
| 4 | 2 Rx-2 Frac-1 Recycle | 7300 | 70% | 2400 | 15% | 3911 | 93% |
| 5 | 2 Rx-2 Frac-2 Recycle | 7300 | 70% | 2357 | 12% | 3944 | 94% |

The new configurations can be applied to grass roots or polygas, MTBE or other available units that can be retrofitted into these configurations. They can be standalone units or can be located upstream of alkylation units. The new configurations will allow the selective dimerization of isobutene and/or 1-butene, and/or propene compounds with the unreactive (or less reactive) olefinic compounds sent to an alkylation unit downstream of the process. The differing conditions in each stage may be used to oligomerize the more reactive olefins such as iso-butene in the first reactor under favorable conditions, as shown in Example 1 below, while passing the less reactive olefins such as 1-butene to the second stage reactor for oligomerization under a more forceful set of conditions appropriate to that feed component. These new configurations in combination with an alkylation unit provide great flexibility to the plant operation. A C₄ LPG feed can be selectively dimerized in one of the new configurations, and the unreacted feed can be sent to the alkylation unit. For example:
1. LPG feed containing C₃ and C₄ compounds; can selectively dimerize propene and isobutene compounds. The unreacted LPG material can be sent to the alkylation unit.
2. LPG feed containing C₄ compounds can selectively dimerize/polymerize isobutene and/or 1-butene. The unreacted LPG material can be sent to the alkylation unit.
3. In case of operational problem in the alkylation unit, the new configurations can be used to dimerize/polymerize all the LPG olefinic compounds.

The new unit configurations also enable operating requirements to be more easily met. For example, in start up and shut down of the unit, specific procedures need to be performed for control of the olefin content at the inlet of the reactor. During reactor start up, the proportion of olefin in the reactor feed stream relative to inert components of the stream will need to be kept at a level which will avoid excessive temperature rise and the creation of hot spots in the catalyst beds; the recycle ratio may be used in combination with adjustment of fresh feed olefin content to achieve this objective. When stable unit operation has been achieved, the amount of olefin in the fresh LPG feed to the reactor may be gradually increased so as to maintain the desired temperature profile in the catalyst beds. Conversely, during reactor shut down, the recycle ratio relative to the fresh feed can be increased in addition to effecting a decrease in the inlet olefin content.

### EXAMPLE 1

Samples of 80/20 MCM-49 on alumina zeolite quadrolobe catalyst were used for this study. Two cc of the fresh MCM-49 catalyst was loaded into a laboratory scale reactor (1 cm i.d., 15 cm long) with 6 cc of silica carbide diluent using a downflow configuration. The zeolite catalyst was dried at 260° C. (500° F.) for 5 hrs with 2 litres/hr of completely dry N₂ flowing through the reactor. After drying of the catalyst was complete, a LPG gas mixture was introduced at 24° C. (75° F.), 5.4 LHSV, 1035 kPag (150 psig). The LPG gas mixture composition consisted of approximately 12.37 vol % 1-butene, 14.07 vol % Isobutylene, and 73.56 vol % n-butane. Product composition was determined by injection into a 150 m column online GC; samples were analyzed about every 2.5 hours. As the catalyst aged with approximately 6 days on stream, 100% isobutylene conversion and approximately 0.6% 1-butene conversion was observed. The product also showed about 6.6 wt % C8s and about 5.3 wt % C9+. Over the 6 day test, C8 concentration increased while the C9+ total decreased correspondingly. Complete isobutylene conversion was observed throughout the test period at low temperature. Low 1-Butene conversion was seen. Very high selectivity toward the conversion of one feed component (isobutylene) was achieved by adjusting operating conditions to low temperature 24 C (75° F.), representing the conditions that might usefully be employed in the first stage of a two stage unit, with the unreacted 1-butene passed to a second stage for reaction under higher severity conditions.

1. A process unit for the conversion of FCC refinery gas feed stream containing light C₃-C₄ olefins into a gasoline boiling range product which comprises:
at least two reactors each containing a fixed bed of a solid, porous, molecular sieve olefin polymerization catalyst, each reactor having a feed inlet and an effluent outlet, the reactors being serially connected for sequential flow of the olefin feed from one reactor to the next,
a fractionation section connected to the effluent outlets of each reactor to receive the effluent from each reactor.

2. A process unit according to claim 1 in which the molecular sieve olefin polymerization catalyst material comprises a zeolite.

3. A process unit according to claim 2 in which the molecular sieve olefin polymerization catalyst comprises a zeolite of the MWW family.

4. A process unit according to claim 1 in which the olefin condensation catalyst comprises a zeolite of the MCM-22 family.

5. A process unit according to claim 4 in which the olefin condensation catalyst comprises a regenerated catalyst.

6. A process unit according to claim 1 which comprises a first stage reactor having a feed inlet and an effluent outlet connected to the inlet of a first fractionator which is in the fractionation section and which has a feed inlet, a heavy fraction outlet and a light fraction outlet which is connected to the feed inlet of a second stage reactor having a feed inlet and an effluent outlet which is connected to the feed inlet of the first fractionator.

7. A process unit according to claim 6 which includes a recycle conduit connected to the light fraction outlet of the first fractionator and to the feed inlet of the first stage reactor for passing light fraction from the first fractionator as recycle to the first stage reactor.

8. A process unit according to claim 1 which comprises a first stage reactor having a feed inlet and an effluent outlet connected to the inlet of a first fractionator which is in the fractionation section and has a feed inlet, a heavy fraction outlet and a light fraction outlet which is connected to the feed inlet of a second stage reactor having a feed inlet and an effluent outlet which is connected to the feed inlet of a second fractionator in the fractionation section.

9. A process unit according to claim 8 which includes a recycle conduit connected to the light fraction outlet of the second fractionator and to the feed inlet of the second stage reactor for passing light fraction from the second fractionator as recycle to the second stage reactor.

10. A process unit according to claim 9 which includes a recycle conduit connected to the light fraction outlet of the first fractionator and to the feed inlet of the first stage reactor for passing light fraction from the first fractionator as recycle to the first stage reactor.

11. A process for the conversion of an FCC light gas stream containing light C₃-C₄ olefins into a gasoline boiling range product which comprises:
feeding the light gas stream containing the light olefins into the feed inlet of a first reactor containing a fixed bed of a solid, porous molecular sieve olefin polymerization catalyst to polymerize the olefins in the feed under a first set of reaction conditions to form an effluent comprising polymerized product in the gasoline boiling range formed from the light olefins,
passing the first stage reactor effluent to a feed inlet of a fractionation section and fractionating the first stage effluent stream to form a heavy fraction comprising gasoline boiling range product and a light fraction comprising unreacted olefins, passing light fraction from the fractionation section to the feed inlet of a second stage reactor containing a fixed bed of a solid, porous molecular sieve olefin polymerization catalyst to polymerize the olefins in the feed under a second set of reaction conditions which are more severe than those of the first set to form a second stage reactor effluent comprising polymerized product in the gasoline boiling range formed from the unreacted light olefins, passing the second stage reactor effluent to a feed inlet of the fractionation section;
fractionating the second stage reactor effluent in the fractionation section to form a heavy fraction comprising gasoline boiling range product and a light fraction comprising unreacted olefins.

12. A process according to claim 11 in which light fraction from the fractionation section is recycled to the feed inlet of the first stage reactor.

13. A process according to claim 11 in which the fractionation section comprises a first fractionator and a second fractionator and light fraction is fed from the first fractionator to the feed inlet of the second stage reactor and the second stage reactor effluent is fed to a feed inlet of the second fractionator to form the heavy fraction comprising gasoline boiling range product and the light fraction.

14. A process according to claim 13 in which light fraction from the second fractionator is recycled to the feed inlet of the second stage reactor.

15. A process according to claim 14 in which light fraction from the first fractionator is recycled to the feed inlet of the first stage reactor and light fraction from the second fractionator is recycled to the second stage reactor.

16. A process according to claim 11 in which the molecular sieve olefin polymerization catalyst material comprises a zeolite.

17. A process according to claim 16 in which the molecular sieve olefin polymerization catalyst comprises a zeolite of the MWW family.

18. A process according to claim 17 in which the olefin condensation catalyst comprises a zeolite of the MCM-22 family.

19. A process according to claim 18 in which the olefin condensation catalyst comprises a regenerated MCM-22 catalyst.

20. A process according to claim 11 in which the first stage polymerization is carried out at a temperature from 150° to 200° C., a pressure up to 3500 kPag and a space velocity of 5 to 30 hr−1 WHSV and the second stage under conditions of relatively higher severity.
